(19) United States
(12) Patent Application Publication
(10) Pub. No.: US 2021/0299311 A1
(43) Pub. Date: Sep. 30, 2021

(54) Multifunction Disinfection Drone Apparatus and Method

(71) Applicant: Simon Siu-Chi Yu, Oakland, CA (US)

(72) Inventor: Simon Siu-Chi Yu, Oakland, CA (US)

(21) Appl. No.: 16/993,139

(22) Filed: Aug. 13, 2020

Related U.S. Application Data
(60) Provisional application No. 63/100,701, filed on Mar. 26, 2020.

(51) Int. Cl. A61L 2/24; B64C 39/02; A01M 13/00; A41D 13/11; B08B 13/00; B08B 7/00; B08B 5/00; B08B 7/04; A61L 2/10; A61L 2/20

(52) U.S. Cl. CPC A61L 2/24; B64C 39/02; A01M 13/00; A41D 13/11; B08B 13/00; B08B 7/0057; A61L 2101/02; B08B 7/04; A61L 2/10; A61L 2/202; B64C 2201/027; B64C 2201/12; B64C 2201/141; B08B 5/00

(57) ABSTRACT

An autonomous drone operated remotely via wireless networks is instructed by its owner flying about in an unoccupied room to activate a system of selectable air and surface disinfection modules. An air purifier captures particles in ambient air and switches on an oscillating motion tubular shaped cage mounted with a plurality of Ultraviolet C spectrum, long range zoom and focusable lens LED projectors to disinfect virus and bacteria. A fan assisted forced air Ozone generator module speeds up disinfecting inconspicuous spots to destroy hidden viruses. Disinfection is completed after the half-life time of dispensed Ozone and an oxygen recovery module rapidly converts residual ozone still in air back to normal oxygen molecules enabling a shortened waiting time for occupants to return to the treated room. A negative ions module subsequently switches on to refresh the air.

### CROSS-REFERENCE TO RELATED APPLICATION

This application claims priority to Provisional Application 63/100,701 filed Mar. 26 2020 by Simon Siu-Chi Yu and also claims benefit to Provisional application No. 63/101,110 filed Apr. 18 2020, both titled ‘Multi Function Disinfection Drone Apparatus and Method’ each incorporated herein by reference, each in its entirety.

### BACKGROUND OF THE INVENTION

Consumer grade home use UV virus disinfection devices are generally for arms length, close distance application. They are ineffective for disinfecting an average size bedroom due to UV attenuates following the inverse-square-law. That is the effectiveness or intensity of the disinfection changes in inverse proportion to the square of the distance from the source.

Commercial grade institutional UV virus disinfection machines employing high power Mercury arc lamps or pulsed Xenon arc lamps are very expensive and these types of devices rely on reflected lights bounced from walls and ceiling. The pulsed Xenon noises irritate people and some people can't tolerate flashing lights. Their output power attenuation also follows the inverse-square-law. All occupants are ordered to vacate the area to be treated with this technology.

Automated mobile ultraviolet light devices that continuously emit UV-C in the range of 254 nm can be placed in patient rooms after patient discharge and terminal cleaning has been performed. A number of these devices can be set to kill vegetative bacteria or to kill spores. These systems often reduce the VRE and MRSA by four or more log 10, and C. difficile by 1-3 log 10. In one comparative trial, a continuous UV-C light system resulted in lower log reductions than a micro-condensation hydrogen peroxide vapor system. Advantages of the mobile, continuous UV-C light devices include their ease of use, minimal need for special training of environmental services personnel, and unlike hydrogen peroxide vapor systems, the ability to utilize the devices without having to seal room vents or doors. Recently, a prospective, multicenter randomized controlled trial comparing a mobile continuous UV-C light system with standard and other enhanced surface disinfection methods has been completed. Results of the trial should be published in the near future.

A pulsed-xenon device, which does not use mercury bulbs to produce UV light, emits light in the 200-320 nm range. It has been shown to significantly reduce pathogens in patient rooms. The manufacturer recommends placing device in 3 locations in a room with 5-7 min cycles (shorter than with some continuous UV-C systems). While a few studies utilizing the device reported reductions in C. difficile infection, a more recent 8-month study in a large institution found no significant reduction in C. difficile infection rates hospital-wide or on four units with high C. difficile infection rates. One carefully-performed trial which compared the pulsed-xenon system with a continuous UV-C light device found that log 10 reductions of pathogens achieved with the pulsed-xenon system were lower than with the continuous UV-C light device. Additional evaluation of the pulsed-xenon UV system by independent investigators is needed.

### SUMMARY OF THE INVENTION

The UAVD (unmanned aerial vehicle drone) includes a control and communications module comprising an electronic central processing unit (CPU), a wireless communication unit, an electronic camera and audio A/V unit and a bus configured to interconnect all drone modules. The UAVD further includes a navigation module comprising a set of 360 degree obstacle avoidance sensors and positioning unit (GPS) configured to autonomously direct the drone to avoid obstacles while in flight.

The disinfection drone is self sufficiently equipped with batteries for its own propeller motor and dedicated power for its accessories. The drone has adapter brackets to accept a cartridge comprising various modules for various purposes. The removable and configurable modular cartridges comprise a front-end Ultraviolet C spectrum LED light module to disinfect incoming polluted air and to excite the Titanium Oxide coated particle catch plates.

The removable and configurable modular cartridge comprises a set of Titanium Oxide coated electrostatic charged plates in a module to convert bacteria to harmless gas through a photocatalysis reaction. Disinfecting is safe when occupants are present in the area because photocatalysis does not contain UV or ozone. The removable and configurable modular cartridges comprise a set of negative ion emitter modules to refreshing air. The removable and configurable modular cartridges comprise a rear-end Oxygen recovery Ultraviolet C spectrum LED light module to rapidly convert ozone to normal oxygen molecules.

The disinfection drone comprises a set of high voltage charged screens in a module to electrocute flying insects. The drone comprises a set of Ozone generators module to disinfect inconspicuous spots which are infected with viruses and to destroy agricultural pests and control algae growth. The (346 pm) Pico meter ozone penetrates the pest's body and causes plugging in their respiratory organs. The module further comprises an oxygen tank to boost concentration and production of ozone. Another advantage of the Ozone generator module comprises a blower fan housing a set of ozone generator plates. Ozone is forced out by the fast spinning blower fan. The ozone generator module can be detached from the drone body and becomes a handheld disinfection sprayer with invisible ozone.

The drone further comprises a module, tubular shaped cage, mounted with a vacuum tube ultraviolet C spectrum projector to disinfect virus and bacteria. The light irradiation from vacuum tube projectors are reflected and focused with reflectors aimed at infected targets. The drone further comprises a module, tubular shaped cage, mounted with a solid state semiconductor ultraviolet C spectrum projector in coherent and non-coherent irradiation to disinfect virus and bacteria over long range. The non-coherent LED light is focused with adjustable focal lenses.

The coherent Laser diode is focusable with an external focal lens for extended distance. The disclosure disinfects viruses, bacteria, mold, agricultural pests and controls algae growth without chemical spray. The disclosure disinfects the entire room from floor to ceiling with minimum blind spots for applications in a very tight spaces for users who don't have trained skills in flying a drone.

The disclosure further comprises an autonomous mobile robot in addition to the disclosed drone systems. The autonomous mobile robot combines a telescopic screw drive tension pole, temporally fixed on a floor with a ceiling tension spring. A screw drive carriage carries the drone as it flies around governed by a screw thread moving up or down with the drone's propeller vector forces. The room is therefore swept with UV irradiate from floor to ceiling.

The autonomous mobile robot plus drone systems are employed for Upper-Room germicidal UV-C airborne virus disinfection. The autonomous mobile robot combines a motorized screw drive shaft mounted on a motorized floor stand with wheels resting on the floor without ceiling support. A carriage connects the screw drive shaft thereto and the drone flies around governed by the screw thread moving up or down via the drone's propellers vector forces or with the motorized screw drive shaft rotation when the drone detects there are no occupants in the room. When the system detects occupants, the drone raises itself to a ceiling level which is above the occupant's head for continuously disinfecting with UV-C irradiation. The disclosure also includes human avoidance object and face detection to avoid its UV beams irradiating the occupants.

The disclosure further includes the removal of the ozone generator module from the drone becomes a handheld ozone sprayer operates by a worker to blast at the occupant, causing viruses dislodge for disinfection.

This disclosure is directed to using drones to disinfect airborne and surface virus, bacteria, mold, control algae growth and destroy agricultural pests. The disclosure is also used for non liquid and non chemical spray personal disinfection. The drone projects a plurality of tightly focused UV beams in a sweeping pattern along with up-down motions to effectively disinfect viruses in long range as well as at short ranges.

The disclosure solves the output attenuation issues of prior applications and devices since it is able to disinfect a large area without following the inverse-square-law. Occupants are also allowed in the area being treated due to an application of advanced technology in the disclosed multi-function disinfection system.

Other aspects and advantages of embodiments of the disclosure will become apparent from the following detailed description, taken in conjunction with the accompanying drawings, illustrated by way of example of the principles of the disclosure herein.

### BRIEF DESCRIPTION OF THE DRAWINGS

FIG. 1 is a cross section view of a multi-function drone fully equipped with modules for disinfecting polluted air, and a vacuum tube UV-C module for virus, and bacteria disinfection in accordance with an embodiment of the present disclosure.

FIG. 2 is a cross section view of a multi-function drone fully equipped with modules for disinfecting polluted air, and a LED UV-C module for virus, and bacteria disinfection in accordance with an embodiment of the present disclosure.

FIG. 2A illustrates LED UV-C use on the module adjustable to zoom in and focus the beam in a highly concentrated flux for long range virus disinfection in accordance with an embodiment of the present disclosure.

FIG. 2B illustrates the drone hovering on a fixed elevation with the LED UV-C module in a stationary position, with two intense dots of beams shown on the wall in accordance with an embodiment of the present disclosure.

FIG. 2C illustrates the drone hovering on a fixed elevation with the LED UV-C module oscillation creating two lines of beams in accordance with an embodiment of the present disclosure.

FIG. 2D illustrates the drone hovering on a fixed elevation with the LED UV-C module oscillating and activating the vibration motor creating two fully filled lines of beams in accordance with an embodiment of the present disclosure.

FIG. 2E illustrates the motion as described in FIG. 2D and includes the drone flying up and down creating a fully irradiated wall of UV-C disinfection irradiation in accordance with an embodiment of the present disclosure.

FIG. 2F illustrates conventional UV disinfection light intensity attenuation following the inverse-square-law.

FIG. 3 is a cross section view of a multi-function disinfection drone fully equipped with modules for disinfecting polluted air, and a Laser Diode UV-C module for virus, and bacteria disinfection in accordance with an embodiment of the present disclosure.

FIG. 4 is a top view of a multi-function disinfection drone showing polluted air being pulled in from top and also depicts the high voltage screen cage for electrocuting flying insects and showing oxygen tanks in accordance with an embodiment of the present disclosure.

FIG. 5 is a bottom view of a multi-function drone showing refreshed air exhausted out from the bottom and also depicts the vacuum tube UV-C projector and ozone generator in accordance with an embodiment of the present disclosure.

FIG. 6 is a bottom view of the multi-function disinfection drone showing the LED UV-C projector and ozone generator wherein the projectors are electronically addressable to avoid irradiating occupants in accordance with an embodiment of the present disclosure.

FIG. 7 is a bottom view of the multi-function disinfection drone showing the Laser Diode UV-C projector and ozone generator wherein the projectors are electronically addressable to avoid irradiating occupants in accordance with an embodiment of the present disclosure.

FIG. 8 is a module top view including the front-end UV-C LED for exciting the Titanium Oxide coating on electrostatic charged plates in accordance with an embodiment of the present disclosure.

FIG. 9 is a module top view of Titanium Oxide coated electrostatic charged plates for performing a photocatalysis function in accordance with an embodiment of the present disclosure.

FIG. 10 is a module top view of an array of negative ion emitters in accordance with an embodiment of the present disclosure.

FIG. 11 is the module top view of the rear-end UV-C LED lighting for rapidly converting residual ozone back to normal oxygen molecules (ozone decomposing action) in accordance with an embodiment of the present disclosure.

FIG. 12 is perspective view of vacuum tube, tubular shaped cage UV-C module in accordance with an embodiment of the present disclosure.

FIG. 12A is a perspective view of a vacuum tube UV-C bulb with a focusing folded reflector in accordance with an embodiment of the present disclosure.

FIG. 13 is a perspective view of a solid state semiconductor laser diode tubular shaped cage UV-C module in accordance with an embodiment of the present disclosure.

FIG. 13A is a perspective view of a UV-C spectrum Laser Diode without a focusing lens in accordance with an embodiment of the present disclosure.

FIG. 14 is perspective view illustrates an element of ceramic ozone generator with fan assisted and extra injection of oxygen to boost ozone concentration in accordance with an embodiment of the present disclosure.

FIG. 15 illustrates naturally how the Sun's ultraviolet creation and destruction of ozone in earth's stratosphere.

FIG. 16 is a perspective view of an LED UV-C drone, its outer safety screen mesh that protects the high voltage electrified insect screen is removed to show the interior.

FIG. 16A is an open center turntable connecting the drone and projector module in accordance with an embodiment of the present disclosure.

FIG. 17 is a perspective view of an autonomous mobile robot configured for non-aerial wide area disinfection system for people unable to pilot a drone and for use in extremely crowded rooms in accordance with an embodiment of the present disclosure.

FIG. 17A is top view illustrates autonomous mobile robot disinfection system, drone turning direction depending on manipulating the four rotor speed in accordance with an embodiment of the present disclosure.

FIG. 17B is perspective view one of a motorized rotary shadow reflector bounce back light beams to minimize target shadows in accordance with an embodiment of the present disclosure.

FIG. 18 is the perspective view of non-aerial Upper-Room germicidal configuration shows the UV-C projectors are positioned above a person's head level to avoid being irradiated with UV-C in accordance with an embodiment of the present disclosure.

FIG. 19 is a portable disinfection booth for a person being blasted with ozone blended with air from overhead position for rapid disinfection in accordance with an embodiment of the present disclosure.

FIG. 20 is a face mask equipped with an active and passive ozone decomposer filter box in accordance with an embodiment of the present disclosure.

FIG. 20A is a cross section view of the active and passive ozone decomposer filter box in accordance with an embodiment of the present disclosure.

FIG. 20B is an UVC LED stick removable for periodic cleaning and reusable on a new mask in accordance with an embodiment of the present disclosure.

FIG. 21 is an ozone generator module detached from the drone; a worker is spraying with invisible ozone instead of liquid for disinfecting targets in accordance with an embodiment of the present disclosure.

FIG. 21A is a perspective view of a detached portable ozone sprayer equipped with concentrated oxygen and a color flashlight for aiming the target to be sprayed in accordance with an embodiment of the present disclosure.

FIG. 22 is a block diagram showing various operation instructions scheduled on the modules that communicate with the drone in accordance with an embodiment of the present disclosure.

Throughout the description, similar and same reference numbers may be used to identify similar and same elements in the several embodiments and drawings. Although specific embodiments of the invention have been illustrated, the invention is not to be limited to the specific forms or arrangements of parts so described and illustrated. The scope of the invention is to be defined by the claims appended hereto and their equivalents.

### DETAILED DESCRIPTION

Reference will now be made to exemplary embodiments illustrated in the drawings and specific language will be used herein to describe the same. It will nevertheless be understood that no limitation of the scope of the disclosure is thereby intended. Alterations and further modifications of the inventive features illustrated herein and additional applications of the principles of the inventions as illustrated herein, which would occur to a person of ordinary skill in the relevant art and having possession of this disclosure, are to be considered within the scope of the invention.

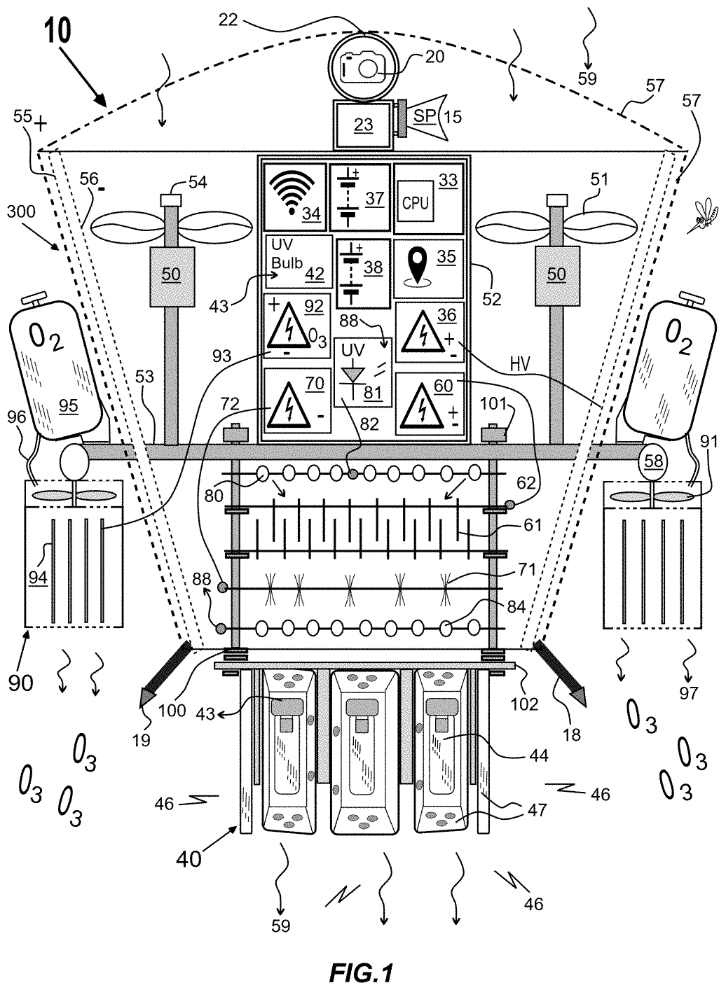

FIG. 1 is the multi function drone 10 equipped with motor 50 which drives propeller 51 installed on the motor shaft 54 in accordance with an embodiment of the present disclosure. The motor 50 is mounted on support frame 53. Drone housing 52 comprises drone computer CPU 33 which communicates with GPS device 35 and Wi-Fi wireless unit 34 and a 360 degree obstacle avoidance device 23 with information feeds from video camera 20. Antenna 22 sends and receives data to its remote controller. A speaker 15 announces a message to warn occupants nearby the area to vacate during the drone in operation. Dedicated battery 37 only serves the drone while battery 38 powers all accessories such as removable and configurable modular cartridge assembly 100 including ultraviolet C generator power supply 42, 81 and high voltage generator modules 92, 60, 36 and 70. The removable and configurable modular cartridge assembly 100 comprises particle collector module 61, front-end UV-C LED module 80, negative ions generator module 71 and a rear-end oxygen recovery UV-C generator 84. The cartridge assembly 100 can be removed if not being used by loosening up mounting nut 101. On the bottom of cartridge 100 is mounted vacuum tube UV-C module 40. The module 40 is a hollow core tubular shaped cage mounted with multiple units of UV-C tubes 44 installed in their focusing reflectors 47. A fan 91 assisted Ozone generator 90 is installed sideways of drone 10 mounted on articulate joint 58 to spray ozone gas in any direction indicated by arrow 97. An oxygen tank 95 feeds concentrated oxygen into the ozone generator 90 to boost Ozone production. Drone 10 also eradicates flying insects such as mosquitoes by equipped cage 300 which includes high voltage screen 55 and 56 protected by an isolated screen 57 guard against accidental electric shock when the cage 300 is electrified.

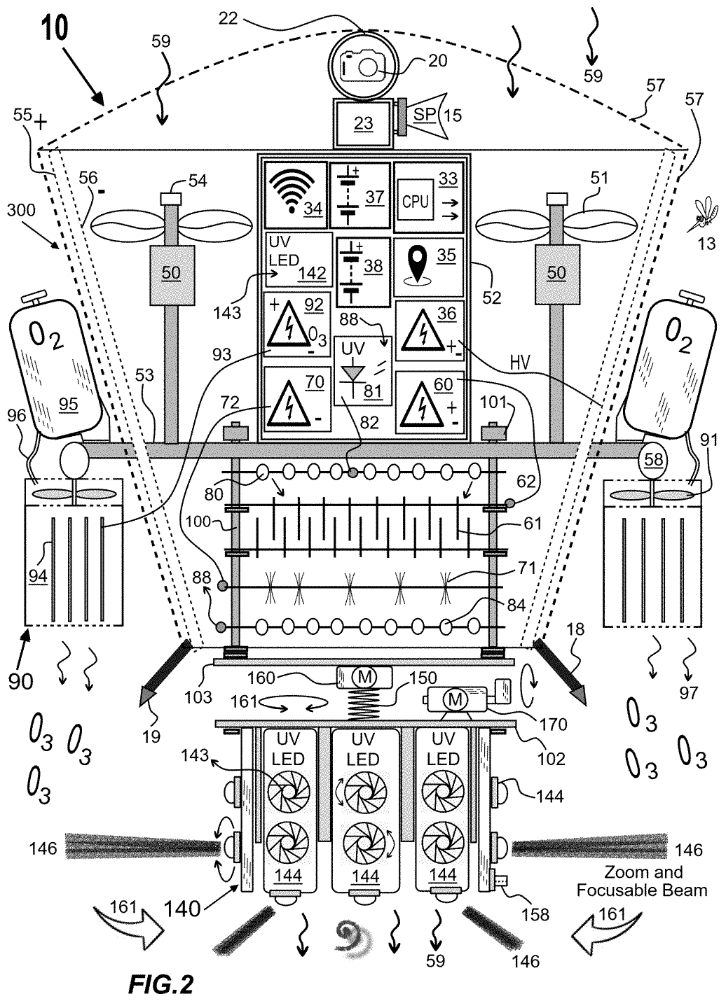

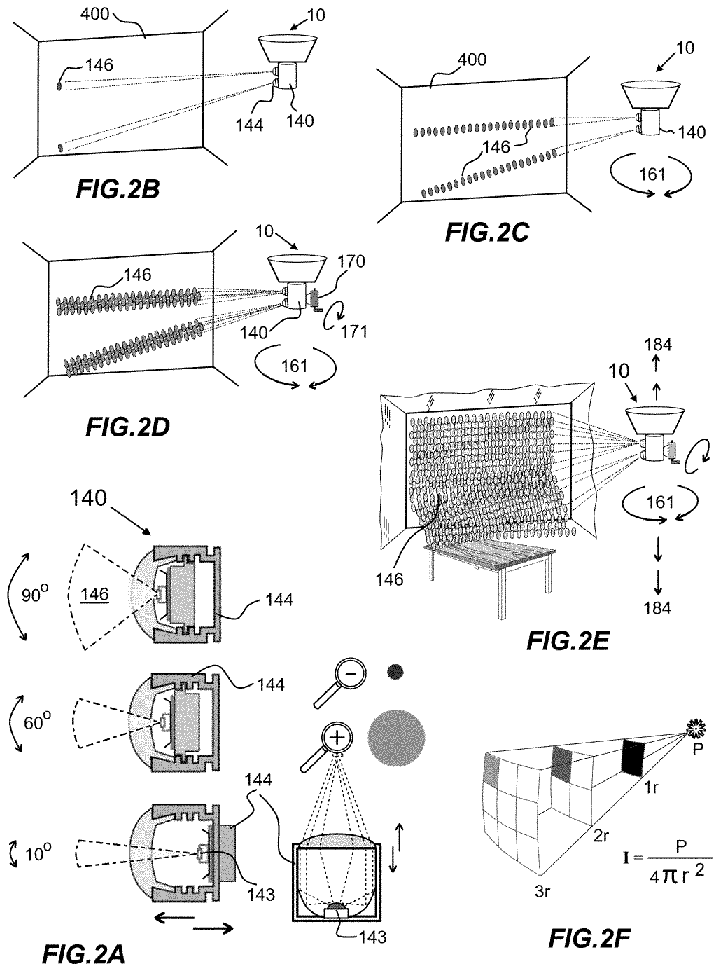

Turning to FIG. 2 is an advanced version of the multifunction drone 10 equipped with motor 50 which drives propeller 51 installed on the motor shaft 54 in accordance with an embodiment of the present disclosure. The motor 50 is mounted on support frame 53. Drone housing 52 comprises drone computer CPU 33 in communication with GPS device 35 and Wi-Fi wireless unit 34 and 360 degree obstacle avoidance device 23 which supplies information from video camera 20. Antenna 22 sends and receives data to its remote controller. A speaker 15 announces a message to warn occupants nearby the area to vacate during the drone in operation. Dedicated battery 37 only serves the drone while battery 38 powers all accessories such as cartridge assembly 100 which includes ultraviolet C generator power supply 142, Bland high voltage generator modules 92, 60, 36 and 70. The removable and configurable modular cartridge assembly 100 comprises particle collector module 61, front-end UV-C LED module 80, negative ions generator module 71 and a rear-end oxygen recovery UV-C generator 84. The cartridge assembly 100 can be removed if not being used by loosening up mounting nut 101. On the bottom of cartridge 100 is mounted with a solid state semiconductor UV-C module 140. The module 140 is a hollow core tubular shaped cage mounted with multiple units of UV-C zoom and focusable LED projector 144. A fan 91 assisted Ozone generator 90 installed on sideways of drone 10 is mounted on articulate joint 58 to spray ozone gas in any direction. An oxygen tank 95 feeds concentrated oxygen via hose 96 into the ozone generator 90 to boost Ozone production. Drone 10 also eradicates flying insects such as mosquitoes by equipping cage 300 with high voltage screens 55 and 56 protected by an isolated screen 57 to guard against accidental electric shock when the cage 300 is electrified.

As described herein, the LED 144 is zooming and focusable, adjustable from wide beam coverage to narrow tight beam irradiation as shown in FIG. 2A. The efficiency of inactivation on a virus depends on the quantity of photons striking the virus. Commercial virus disinfection machines costs hundreds of thousands of dollars and include a single Xenon gas arc lamp or multiple elongated tubes Mercury vapor arc lamps. They are designed to be floor operated and require a very long treatment time for disinfection due to their UV light dispersing omnidirectionally. They use very high output power UV vacuum tubes to compensate the losses due to distance to reach a target. Turning to FIG. 2F illustrates the UV also follows the inverse-square-law of intensity, I=power divided by 4 π times radius square. Their quantities of UV photos reaching the target are in the rate of hyperbolic diminishing returns.

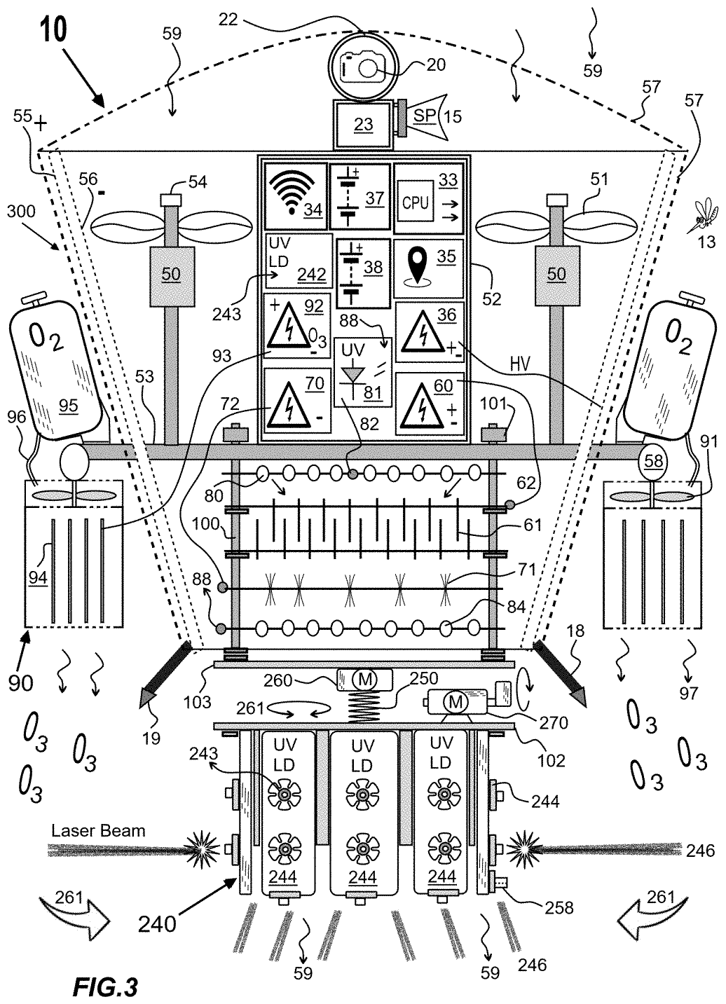

The disclosure uses LED 144 or laser diode 244 to eliminate the physical inverse-square-law disadvantage. Returning to FIG. 2A, the LED 144 used in FIG. 2 is small, very tiny footprint unidirectional point source light which is easy to adapt an optical lens to zoom and focus its light beam pattern. The lens of LED 144 zooms in or out to focus light to a highly concentrated tight photon beam 146 to efficiently inactivate viruses in minimal time. To cover an entire space with UV irradiation, turning to FIG. 2B as illustrated, the drone 10 hovers on air and includes two tightly focused UV beams 146 striking on wall 400. Turning to FIG. 2C, the drone hovers while switches on motor 160 rotate the LED module 140 as indicated by arrow 161 for two lines of UV beams 146 striking the wall 400. The line of beam 146 is placed on pulse mode for illustration purpose; a continuous line of light 146 is preferred. Then in FIG. 2D with the same condition as FIG. 2C, switches on the vibration motor 170 indicated by arrow 171. The two lines of UV beam 146 further fill and over scan without missing any void. To fully irradiate the wall 400 with UV, the drone 10 flies in ascent and descent motions indicated by arrow 184 between elevations from floor and ceiling as illustrated on FIG. 2E. Using zoom and focusing UV beam via an LED as illustrated in FIG. 2, or a laser diode version in FIG. 3 enable disinfection in long range with shorter time and each pixel of beam remaining as powerful as from the light source to the target. The laser diode 244 version is ideal for large space disinfection with an optical focus lens attached in front of the diode 244.

Turning to FIG. 3 is another version of the advanced multi function drone 10 equipped with motor 50 driving propeller 51 installed on the motor shaft 54 in accordance with an embodiment of the present disclosure. The motor 50 is mounted on support frame 53. Drone housing 52 comprises drone computer CPU 33 in communication with GPS device 35 and Wi-Fi wireless unit 34 and 360 degree obstacle avoidance device 23 which provides information from video camera 20. Antenna 22 sends and receives data to its remote controller. A speaker 15 announces a message to warn occupants nearby the area to vacate during the drone operation. Dedicated battery 37 only serves the drone while battery 38 powers all accessories such as cartridge assembly 100 which includes ultraviolet C generator power supply 242, 81 and high voltage generator modules 92, 60, 36 and 70. The removable and configurable modular cartridge assembly 100 comprises particle collector module 61, front-end UV-C LED module 80, negative ions generator module 71 and a rear-end oxygen recovery UV-C generator 84. The cartridge assembly 100 can be removed if not being used by loosening up mounting nut 101. On the bottom of cartridge 100 is mounted a solid state semiconductor UV-C module 240. The module 240 is a hollow core tubular shaped cage mounted with multiple units of UV-C solid state Laser diode (LD) projector 244. A fan 91 assisted Ozone generator 90 installed sideways of drone 10 mounted on articulate joint 58 sprays ozone gas in any direction. An oxygen tank 95 feeds concentrated oxygen via hose 96 into the ozone generator 90 to boost Ozone production. Drone 10 also eradicates flying insects such as mosquitoes via a cage 300 which includes high voltage screens 55 and 56 protected by an isolated screen 57 to guard against accidental electric shock when the cage 300 is electrified.

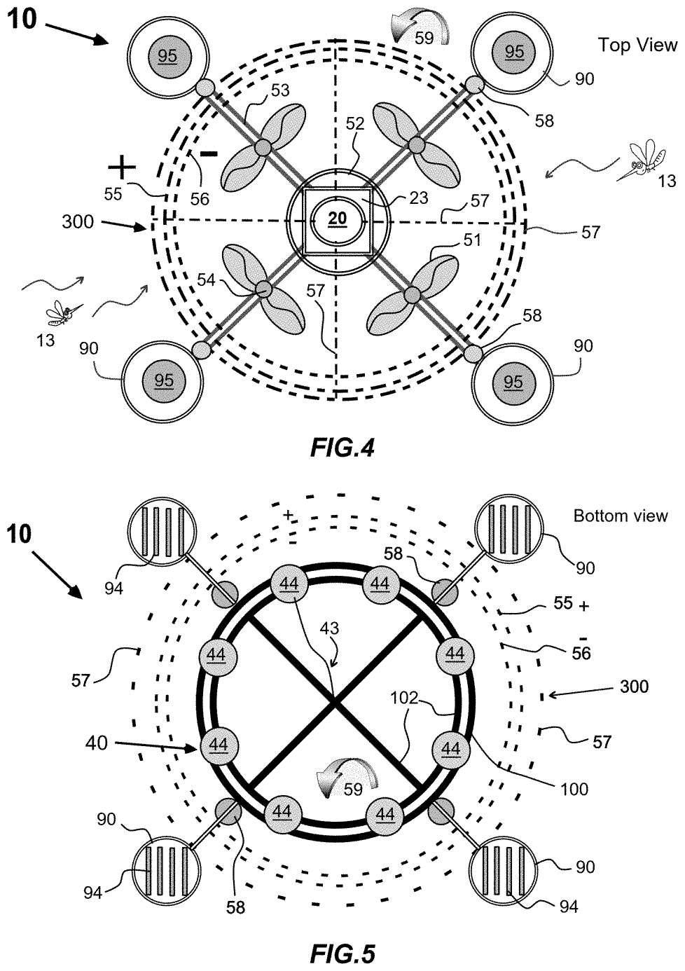

FIG. 4 shows a top view of drone 10 in accordance with an embodiment of the present disclosure. An electrified high voltage cage 300 comprises oppositely charged screen mesh 55 and 56. Flying insects such as mosquitoes 13 contact the screens 55, 56 and are immediately zapped to death. The safety protection screen 57 extends from the drone's 10 body to the top thereof. Also shown is ozone generator 90, its oxygen tank 95 and its injection hose 96. Air current pulls into top of drone 10 as indicated with arrow 59.

FIG. 5 shows the bottom view of the vacuum tube UV-C drone 10 in accordance with an embodiment of the disclosure. Shown are cartridge assembly 100, a cartridge mounting cage 102 mounted with multiple units of vacuum tube UV-C bulb 44 around hollow core tubular shaped cage module 40, power supply cable 43 connect to power supply 42 and articulate joint 58 which allows ozone generator 90 to aim at any angle. Inside the ozone generator 90 there are ceramic ozone discharge plates 94. Purified air exhaust flow from bottom of drone 10 is indicated with arrow 59.

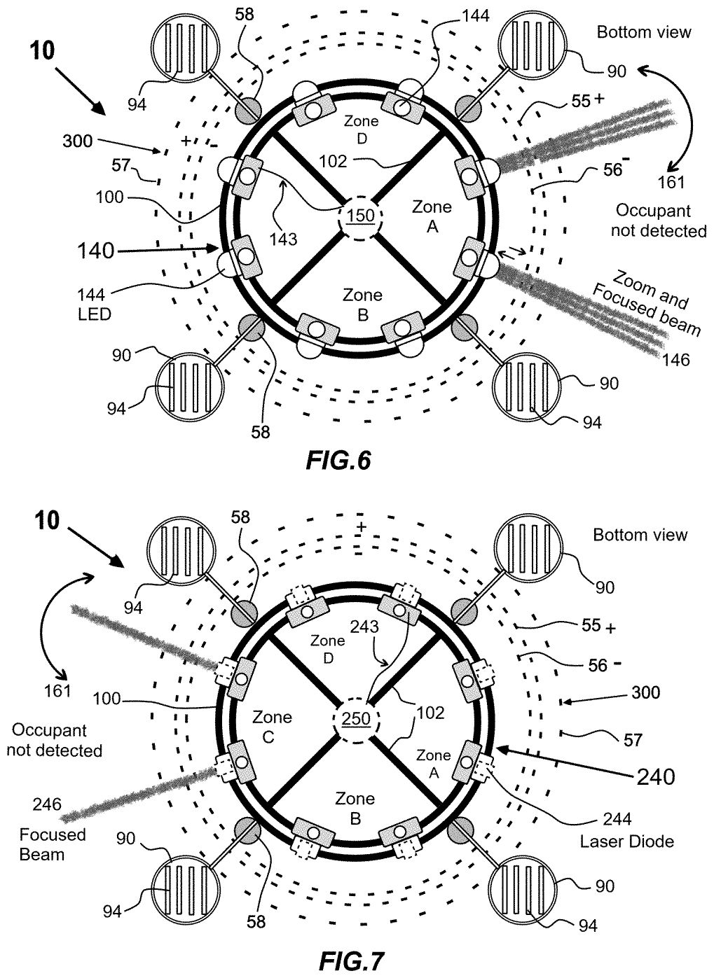

Turning to FIG. 6 is shown the bottom view of solid state semiconductor LED UV-C drone 10 in accordance with an embodiment of the present disclosure. Shown are cartridge assembly 100, a cartridge mounting cage 102 mounted with multiple units of solid state UV-C LED projector 144 around hollow core tubular shaped cage module 140, power supply cable 143 connected to power supply 142 and articulate joint 58 which allows ozone generator 90 to aim at any angle. Inside the ozone generator 90 there is located a ceramic ozone discharge plate 94.

There are plurality of UV projectors 144 installed on the outer surface of tubular shaped cage module 140. The projectors 144 are electronically partitioned in addressable array configurations. The arrays communicate with the drone camera 20 to perform live object and face detection of human presence. A specific array which registered a human presence corresponding to camera 20 captures an image switched off accordingly to avoid the occupants being irradiated with UV-C beams 146 from the specific projectors 144 in the areas. The UV-C beams 146 in the arrays are human avoidant and pre-programmed for a sequence of actions for the cage module 140 to perform and are self directed to avoid occupants being irradiated. FIG. 6 shows the camera 20 for detecting humans in zones B, C and D. The projectors 144 are active only in Zone A in this embodiment.

Turning to FIG. 7 is shown the bottom view of a solid state semiconductor laser diode UV-C drone 10. The cartridge assembly 100, a cartridge mounting cage 102 mounted with multiple units of solid state UV-C laser diode projectors 244 around the hollow core tubular shaped cage module 240, power supply cable 243 connected to power supply 242 and articulate joint 58 allow ozone generator 90 to aim at any angle. Inside the ozone generator 90 there is ceramic ozone discharge plate 94.

There are plurality of UV projectors 244 installed on the outer surface of tubular shaped cage module 240. The projectors 244 are electronically partitioned in an addressable array configuration. The arrays communicate with the drone camera 20 which performs live object and face detection of human presence. A specific array registers human presence that corresponds to camera 20 capturing an image switched off accordingly to avoid the occupants being irradiated with UV-C beams 246 from the specific projectors 244 in the areas. The UV-C beams 246 in the arrays are human avoidant and pre-programmed with a sequence of actions for the cage module 240 to perform and self direct to avoid occupants being irradiated. FIG. 7 shows the camera 20 detecting a human in zone A, B and D. The projectors 244 are active only in Zone C in the present embodiment.

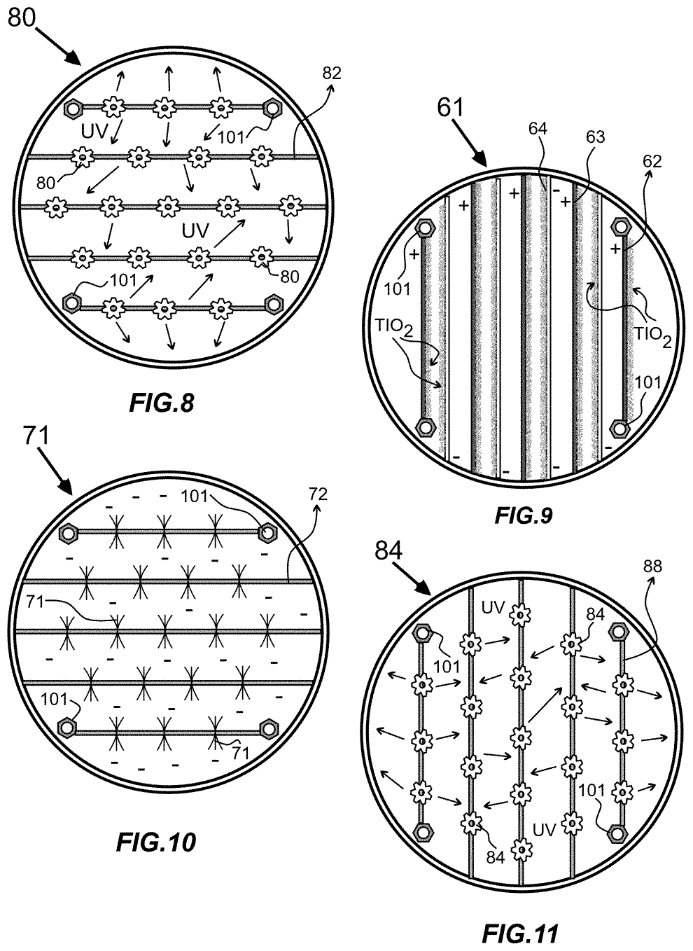

FIG. 8 is the top view of a front-end LED UV-C light module 80 taken out from removable and configurable modular cartridge 100. The UV-C light disinfects the passing air pulled in from propellers. The UV-C is also used to excite TIO2 coatings on the surfaces of particle collector plates.

FIG. 9 is the top view of spaced apart electrostatic TIO2 coated plates per module 61 taken out from cartridge 100 in accordance with an embodiment of the present disclosure. The titanium oxide is a metal oxide semiconductor coated on plates 63 and 64 configured to convert polluted air to harmless CO2 and water via a photocatalysis reaction by irradiating UV light from module 80.

FIG. 10 is a top view of a negative ions generator module 71 taken out from a cartridge 100 in accordance with an embodiment of the present disclosure. The negative ions help polluted air particles particulate on the floor and refresh the room air.

FIG. 11 is a top view of a rear-end LED UV-C light module 84 taken out from a cartridge 100 in accordance with an embodiment of the present disclosure. The 240 nm to 340 nm UV-C light function is for rapidly converting residual ozone still in the air back to normal oxygen molecules; it is a simulated method to decompose ozone.

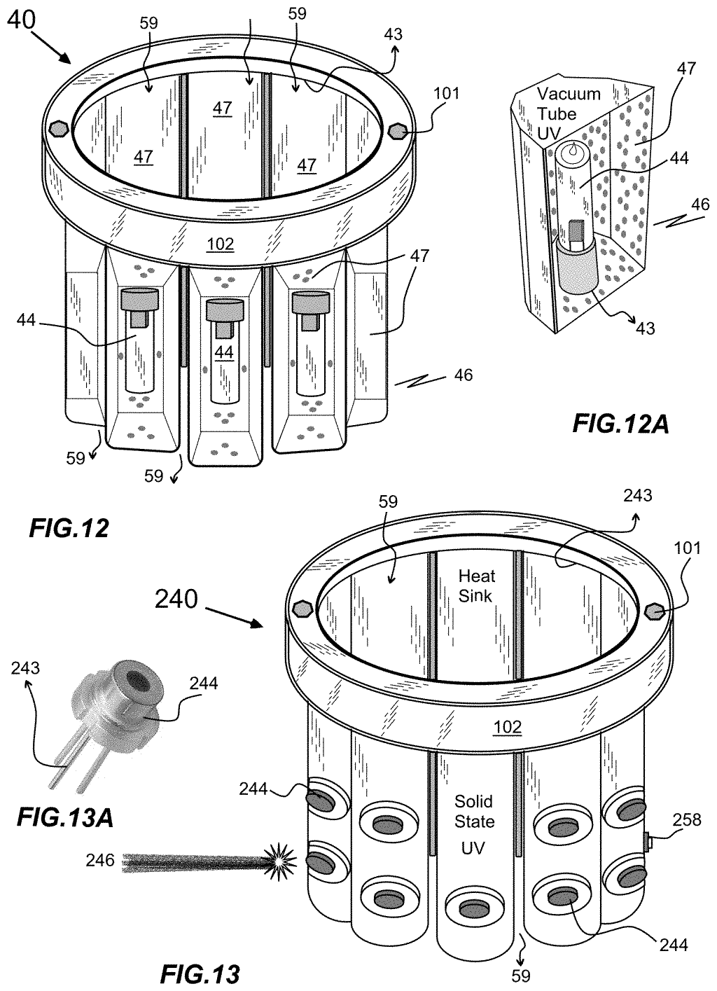

Turning to FIG. 12 is a perspective view of a vacuum tube UV-C module 40 in accordance with an embodiment of the present disclosure. The module 40 is a hollow core, tubular shaped cage attached to a mount 102. The mount 102 is bolted on a bottom of drone 10 cartridge 100 through the hardware nut 101. Multiple units of vacuum tube UV-C bulbs 44 are installed on the surface area of module 40. Light beam 46 is shown irradiated rather than a tight beam.

FIG. 12A showing one of UV-C bulb 44 installed in a focusing reflector 47 to boost its light output efficacy in accordance with an embodiment of the present disclosure.

In FIG. 13, a perspective view of a solid state semiconductor Laser Diode UV-C module 240 is illustrated in accordance with an embodiment of the present disclosure. The module 240 is a hollow core, tubular shaped cage attached to mount 102. The mount 102 is bolted on bottom of drone 10 cartridge 100 through the hardware nut 101. Multiple units of solid state semiconductor Laser Diode UV-C projectors 244 are installed on the surface area of module 240. Light beam 246 irradiates a pattern shown via a non focused coherent beam for a wider spread of light coverage. A small focusing lens can be attached in front of the diode 244 enabling the laser to become coherent and focused for very long range disinfection application. The resulting focused and coherent laser beam is very powerful and users should wear eye protection gear since UV-C is invisible to humans. A visible red 650 nm laser pointer diode 258 is added to guide the scanning area and signaling no human traffic is allowed in the area. The projectors 244 are grouped and assigned to different arrays for addressing and partitioning.

FIG. 13A shows one of UV-C laser diodes 244 without a focusing lens in accordance with an embodiment of the present disclosure.

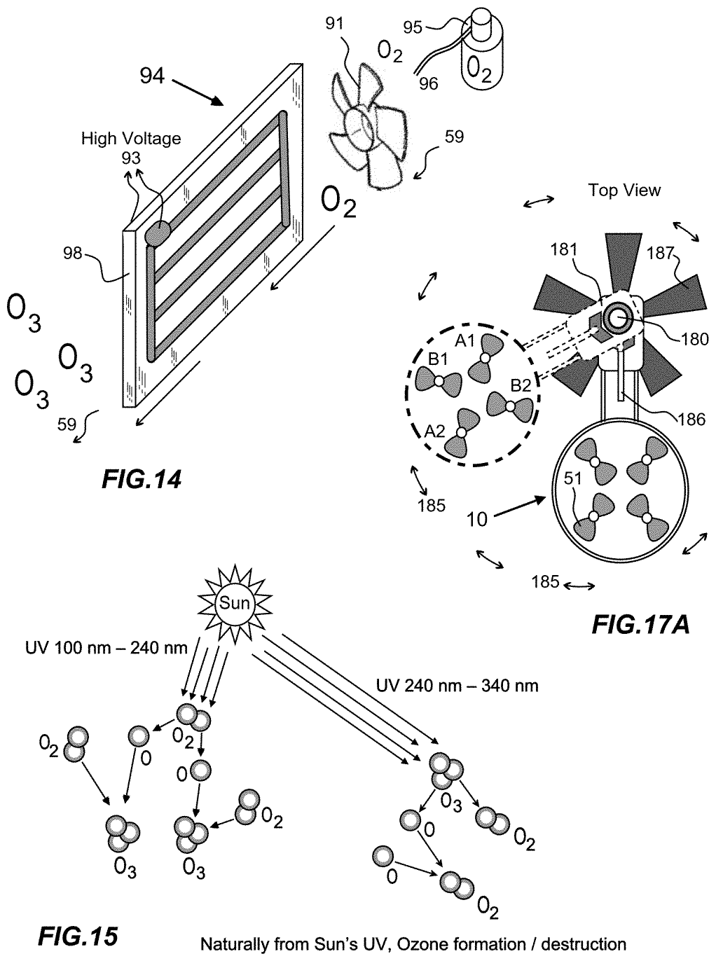

Turning to FIG. 14 is the perspective view of ozone generation element 94, a corona discharge ceramic plate 98 taken out from ozone generator module 90 in accordance with an embodiment of the present disclosure. A fan 91 pulls in natural air 59 which contains 21% of the oxygen passing element 94 to become ozone. An oxygen tank 95 injects extra pure oxygen via hose 96 to boost ozone level. The kinetic diameter of ozone is slightly larger than 346 Pico meters.

As it is gas, its able to seep into gaps and be very powerful to disinfecting viruses hidden in inconspicuous space. Naturally the sun irradiates a broad spectrum of UV light that creates ozone and restores ozone to normal oxygen molecules in the Stratosphere.

FIG. 15 illustrates oxygen molecules, O2 in the upper stratosphere absorbing short wavelength UV radiation between 100 nm to 240 nm dissociating into ozone in accordance with an embodiment of the present disclosure. Ozone, O3 is a strong absorber of longer wavelength electromagnetic radiation between 240 nm to 340 nm. The absorbed energy converts ozone into normal oxygen molecules O2. The disclosure uses the rear-end oxygen recovery module 84 comprising an array of UV-C about 265 nm LED to rapidly convert residual ozone passing through module 84 pulled in from the propeller blades 51 of drone 10.

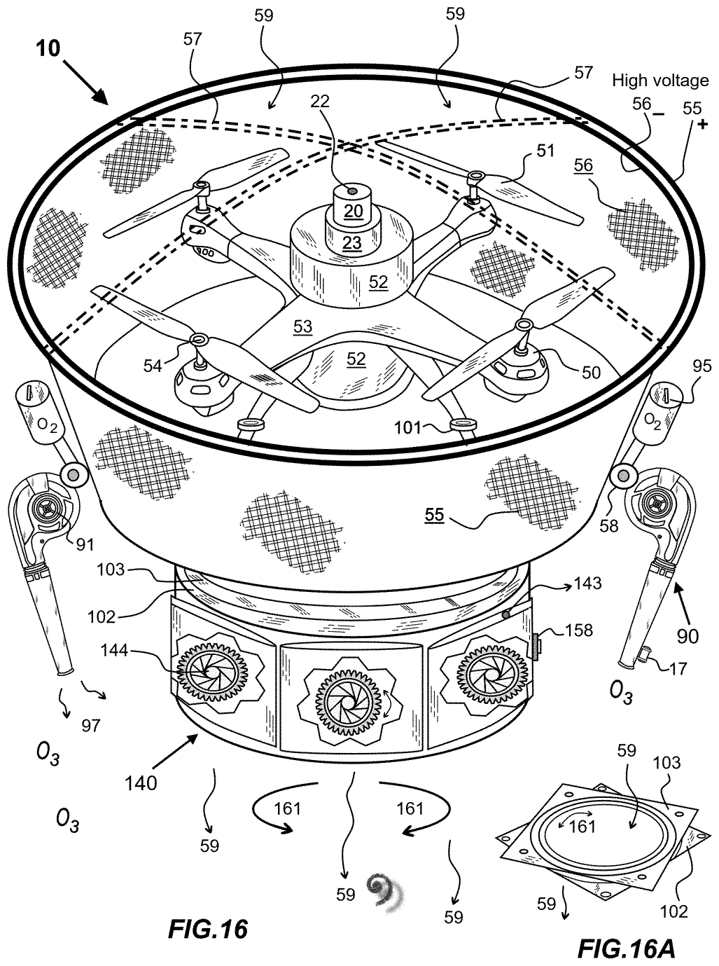

Turning to FIG. 16 is the perspective view of drone 10 as shown and described in FIG. 2. The safety protection screen 57 is removed to show only two cross bars to remind users of safety features. The cage 300 is shown comprising two layers of electrified high voltage screens 55, 56. Insects, such as mosquitoes 13 carrying diseases including dengue virus are electrocuted upon contact with the cage 300. A set of fans 91 assist forced air ozone modules 90 attached on articulated joints 58 directing ozone in any direction. Pure oxygen stored in tanks 95 is optional for boosting ozone production level. A color flashlight 17 is mounted on the nozzle of ozone generator 90 for warning ozone is being sprayed because ozone is invisible to humans. On bottom of drone 10 is mounted a bi-directional rotating motion motor 160 indicating with arrow 161 that module 140 is installed with multiple units of solid state semiconductor UV-C LED 144. The LED 144 zooms and is focus adjustable from wide beam coverage to a narrow tight beam as shown on FIG. 2A. FIG. 16A is an open center turntable 102, 103 used for connecting drone 10 and the projector module 140.

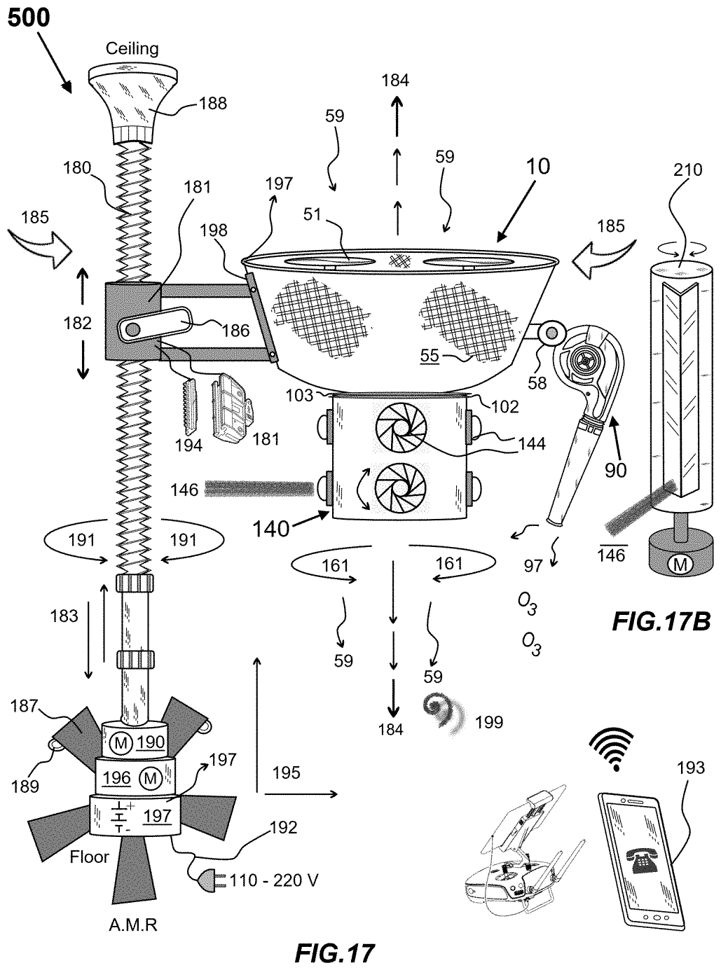

Turning to FIG. 17 is illustrated an autonomous mobile robot plus a drone 10 configured for wide area disinfection system 500 for users who have no knowledge of flying a drone 10 in a very crowded tight space. The drone 10 is equipped with LED UV module 140. The drone 10 further comprises a detachable stab-in mount bracket 198 connected to a screw drive carriage 181. The carriage 181 includes a threaded rack 194 which can engage to the telescopic tension screw drive shaft 180. The disengage lever 186 is shown in a position relative to the carriage 181 engaged with the threads of the screw drive pole 180. The top of screw drive shaft 180 can be temporarily fixed to the ceiling by a bumper 188 for a more secure sturdy support from tipping over, or just using its motorized floor stand 187 without ceiling support. When the system 10 is turned on, the drone 10 tends to rotate depending on the force differential. For example, as shown in FIG. 17A is the top view of a drone 10. There are four Propeller blades 51 controlled by its system gyro. If Blades A1, B1 spin faster than A2, B2, the drone 10 steers in a clockwise direction shown by arrow 185. Likewise, if blades A2 and B2 spin faster than A1 and B1, the result is steering counter-clockwise. Drone 10 remains still when all blades 51 are at the same speed. The levers 186 lock the threaded rack 194 of carriage 181 onto the screw drive 180. The drone 10 will follow the threads pattern of screw drive 180 to rotate in a direction indicated by arrow 185 either going up or coming down in a controlled manner. To bring the drone 10 to a particular height, a user simply activates the screw drive shaft motor 190 while keeping the drone 10 in a still position. Another alternative is to simply twist the lever 186 clockwise to disengage the threaded rack 194 to free carriage 181. The drone 10 now can slide up and down freely along the screw drive shaft 180 as shown by arrow 182.

FIG. 17B is one of the motorized rotary shadow reflectors 210 which bounce back forward going UV light beam 146 to reach the backside of a target. The rotating reflector 210 includes a plurality of shiny mirrors, but without reflector 210 the backside of the target will not be disinfected. It is important to let the drone 10 fly in ascenting and descenting motions through the two sets of counter-rotating blades 51 to create air turbulence 199. This turbulence action 199 dislodges dust, virus and the like on airborne surfaces. It is sucked in from top of drone 10 passing a series of treatment modules 80,61,71,84 then exits from bottom of drone 10 indicated by wind arrow 59. Other than using screw drive shaft 180, the shaft 180 can use a belt drive or chain drive or linear actuator 653 to serve the same purpose.

The motorized floor stand 187 includes extendable legs for a taller screw drive shaft 180 to add stability. The screw drive shaft 180 can be driven by stepping motor 190 to rotate the shaft 180 shown in arrow 191. A ring nut 183 adjusts the height of the shaft to maximize the effectiveness of the system 500. The system 500 further includes a motor 196 to drive the floor stand 187 on wheels 189 autonomously as indicated by direction 195 via the drone 10 navigation system 20, 22, 23, 33, 34 and 35. A battery pack 197 provides power for the system 500. Utility power 192 is available for recharging the battery 197. Since the system 500 should operate in an unoccupied room, it also includes human avoidant features. A smart phone 193 is used to remote control the system 500 movements. Users can monitor the operation with FPV (First-Person View) via Wi-Fi from a smartphone.

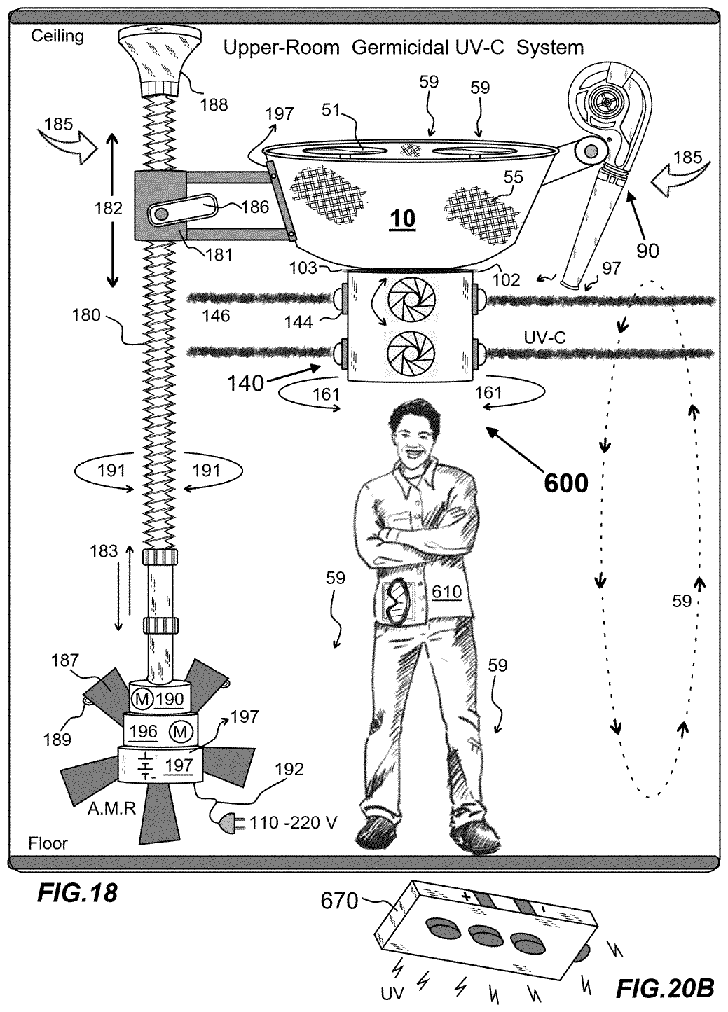

In FIG. 18 is an Upper-Room germicidal overhead disinfecting autonomous mobile robot plus a drone 10 system 600 in accordance with an embodiment of the disclosure. Illustrated here is circulating still air in a room with the help of drone 10 and propeller 51 downdraft indicated by arrow 59. The drone's propellers 51 create a convection air current so that polluted air and viruses are pulled into the drone 10 to be purified by the cartridge module 100. The viruses raised to a ceiling level will be inactivated by UV-C module 140. The upper-room disinfection with UV-C is useful in almost any situation in homes, schools, churches, restaurants and more. When the camera 20 detects an occupant, the human avoidant object and face detection drone 10 will automatically rise to ceiling level about seven feet from the floor. The UV-C beams 146 irradiate above occupant's head assured by the camera 20. After the occupant leaves the room, the system 600 starts irradiating the vacant room from floor to ceiling.

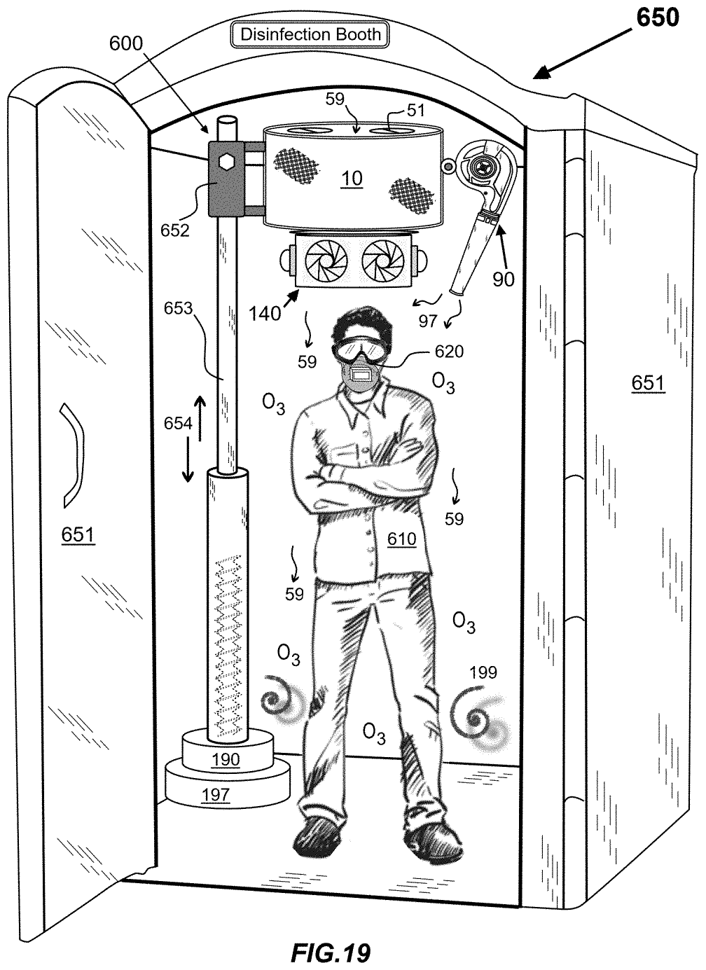

Turning to FIG. 19 is a personal portable isolation ozone disinfection booth system 650 constructed of plastic panel booth 651 or a lightweight pop-up enclosed canopy in accordance with an embodiment of the present disclosure. The user 610 protects themselves with active ozone filtering mask 620 comprising UV LED ozone decomposer 660 lined with manganese oxides or activated carbon 690 in padding. A commercially available mask from 3M Particulate Respirator 8514, N95 and eye protection gear can substitute the mask 620 shown here to keep the user safe by avoiding excessive exposure to ozone. The portable disinfection booth system 650 illustrates the motorized floor stand 187 is not needed. A simpler two sections or multi sections conceal the screw drive shaft linear actuator 653 instead of the sample shown in FIG. 18 via an exposed single piece high reach long screw drive shaft 180 for high ceiling application. However, care should be taken in considering safety that wind turbulence 199 can lift a person's long hair to catch in the exposed turning screw drive shaft 180. The bottom section of actuator 653 is stationary. The screw drive motor 190 provides the ascent and decent motion to drone 10 via drone mounting bracket 652 shown on arrow 654. The system 650 can be placed in the front porch on a house or door entrance of a school to disinfect each individual prior to admitting entrance to the interior of respective establishments will benefit everyone's health. The sample linear actuator 653 when fully extended reaches nine feet (2.7 meter) tall and contracts to a much shorter length for ease of transport.

The forced air assisted ozone generator 90 can be detached from drone 10 for use as a portable disinfectant sprayer unlike the conventional liquid mist sprayer fogging with chemicals. The ozone generator 90 sprays ozone instead to disinfect viruses and can be used to spray high concentrations of ozone to destroy farm pests.

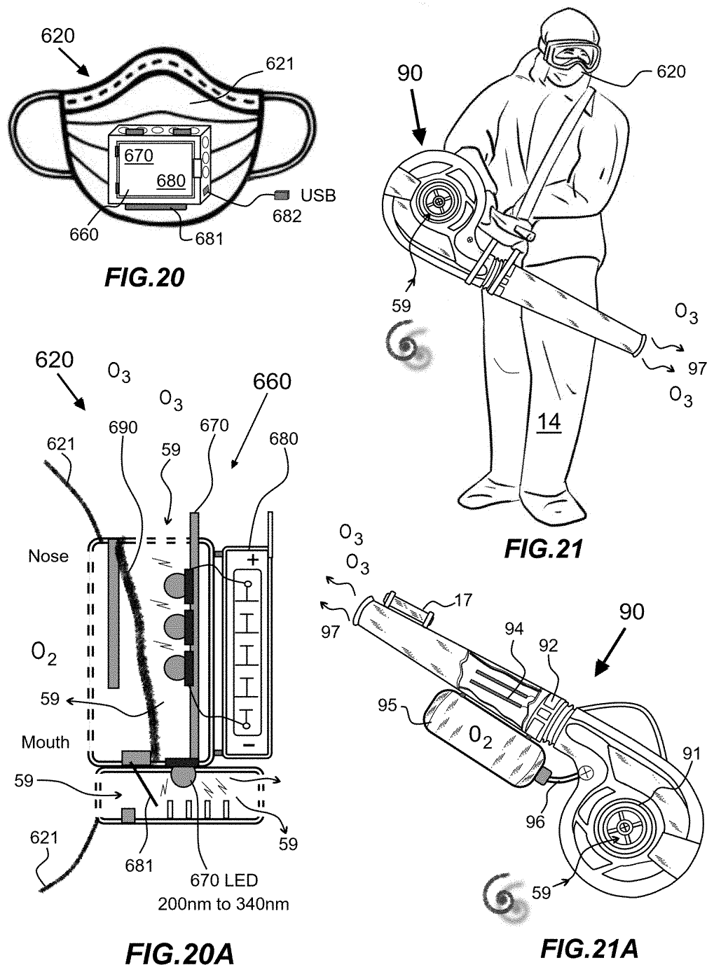

FIG. 20 displays the mask 620 used in FIG. 19 worn by the occupant 610 in accordance with an embodiment of the present disclosure. FIG. 20A is a side exposed view of mask 620 showing fabric 621 and battery 680 which powers the reusable ozone decomposing UV LED stick 670 shown on FIG. 20B over wavelengths 200 nm to 340 nm. A passive activated carbon ozone filter 690 installed in slanted position slows down air velocity to provide added safety to when LED 670 fails. Airflow 59 shown in FIG. 20A mixed with residual ozone enters into the decomposer box 660 irradiated with UV LED 670 to decompose ozone to stable diatomic oxygen (normal oxygen molecules). An exhale check valve compartment 681 provides moisten fluid to be irradiated with UV LED 670 prior to exiting the mask 620. The passive filter 690 is made from activated carbon, manganese oxides and the like. An USB socket 682 picks up power from a smart phone or another portable power source to greatly extend the run time of the mask 620.

The illustration in FIG. 21 depicts the ozone generator 90 removed from drone 10 in accordance with an embodiment of the present disclosure. A worker 14 performs ozone spray for virus disinfection. FIG. 21A shows the ozone sprayer 90 carrying a small oxygen tank to boost ozone output to increase its effectiveness. A color flashlight 17 can be red or green mounted on the nozzle of the ozone generator 90 to guide the worker 14 to aim the invisible ozone. Ozone will decompose into normal oxygen molecules in about fifteen minutes in open air.

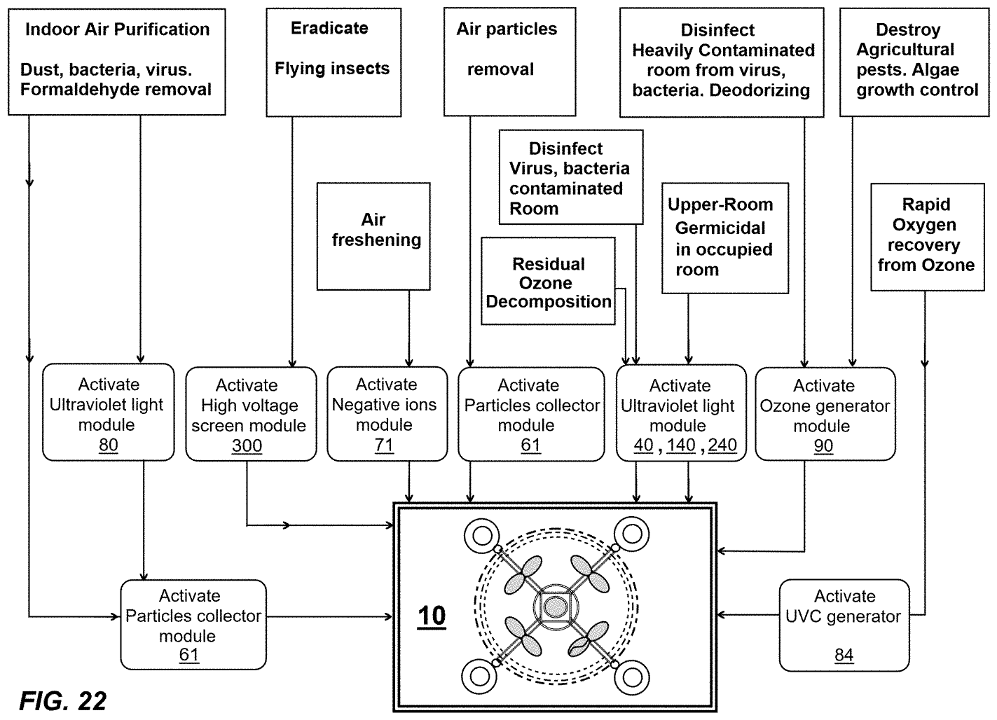

FIG. 22 illustrates the various functions the drone 10 can perform in accordance with an embodiment of the present disclosure. The invention installs a rear-end UV-C light about 265 nm in wavelength into module 84 with the help from drone's propellers 51 to re-circulate the residual ozone that is still floating on air rapidly converted back to normal harmless oxygen. The rear-end UV-C module 84 shortens the half-life of ozone decomposition time, thus allowing occupants to return to the treated area in shorter wait time for their health safety. The disclosure further utilizes the module ozone 90 to destroy agricultural pests such as caterpillars via a high concentration of ozone to plug the insect's respiratory system cause damage to their internal organs. An alternative to flexible drive shaft 150 and 250 shown in FIG. 2, 3 is substituted with an open center turn table shown in FIG. 16A integrated to adapter ring 103 and the mount 102 of UV module 40, 140 and 240 to provide oscillation motion. The disclosure uses object and face detection to protect occupant from irradiation of UV-C. Focused UV-C beams can be continuous operated in the upper-room configuration.

What is claimed is:

1. An unmanned aerial vehicle drone (UAVD), comprising:
at least one projector configured for an ultraviolet spectrum, collimated electromagnetic radiation and infectious disease disinfection to inactivate bacteria and virus via an optical lens and reflector producing a low divergence narrow angle energy beam directed to strike a target over a variable distance and a long-range;
a detachable fan and ozone generator configured to force air and blast a disease infected area with a mixture of air and ozone for rapid disinfection, blast a person in a closed booth for isolated disinfection, blast a person for disinfection in an open-air setting and in an enclosed structure, blast ozone with an optional extra feed of pure oxygen to boost ozone concentration for destroying agricultural pests in farms;
a control and communications module comprising an electronic central processing unit (CPU), a wireless communication unit and an electronic camera and an audio A/V unit;
a navigation module comprising a set of 360 degree obstacle avoidance sensors and a positioning unit (GPS) configured to autonomously direct the drone to avoid obstacles while in flight;
a bus configured to interconnect the projector and ozone generator, the control and communications module and the navigation module; and
a modular cartridge in a removable and configurable relation to the UAVD and an electromechanical docking port for the modular cartridge with the drone, the modular cartridge therefore in electromechanical communication with the drone.

2. The unmanned UAVD of claim 1, wherein the projector comprises an ultraviolet vacuum tube gas-discharge lamp aided by a folded sheet reflector configured to direct a unidirectional light to improve the projector's efficacy.

3. The unmanned UAVD of claim 1, wherein the projector comprises an ultraviolet solid state semiconductor Light Emitting Diode (LED) configured to project a low divergence narrow angle beam aided by a variable focal length collimator and lens.

4. The unmanned UAVD of claim 1, wherein the projector comprises an ultraviolet solid state semiconductor laser diode (LD) configured to project a non-focus coherence wide beam for a close distance disinfection.

5. The unmanned UAVD of claim 1, wherein the projector comprises an ultraviolet solid state semiconductor laser diode (LD) configured to project a focused coherence narrow tight beam aided by a collimator and lens for a long range disinfection.

6. The unmanned UAVD of claim 1, wherein the modular cartridge further comprises a variety of functional modules for specific application including a front-end UV light, titanium oxide coated electrostatic particle collector plates, negative ion emitters, rear-end rapid oxygen recovery UV light, wherein front-end UV light disinfects incoming air pulled in from spinning drone's propellers and irradiates the titanium oxide to effect photocatalysis reaction to disinfect airborne viruses, an array of rear-end ultraviolet light in wave length between 200 nm to 340 nm rapidly decomposing residual ozone which is exiting from the unmanned UAVD returning to normal oxygen molecule, two sections motorized ball bearing open center turntable with its upper section mounted to bottom of the modular cartridge and an adapter mounted with vibrator motor and a motorized flexible drive shaft to substitute the use of the turntable.

7. The unmanned UAVD of claim 1, further comprising a hollow core tubular shaped cage module attached to a lower section of motorized turntable enabling the cage to oscillate or rotation motions, a plurality of UV projectors installed on the outer surface of the tubular shaped cage module, the projectors electronically partitioned in addressable arrays configured to communicate with the camera to perform live object and face detection of human presence via a camera captured image to switch off UV-C beams to avoid the occupants being irradiated in the specific areas, wherein the tubular shaped cage defines an open center allowing high velocity air downdraft passing through specific purpose modules installed on the removable modular cartridge.

8. The unmanned UAVD of claim 1, wherein the projectors are carried by a hollow core tubular shaped cage in an oscillation or rotation motion projecting a line of low divergence narrow angle beam of Ultraviolet rays striking onto a target in a continuous operating mode or projecting a line of a dotted beam in a pulse mode, the projectors irradiate the target with a plurality of repeated and progressing lines of ultraviolet beams based on the unmanned UAVD flying in ascent and decent motions in conjunction with a vibrator motor and an oscillating or rotating motion of a tubular shaped cage and a motorized rotary shadow reflector enhances light beams bouncing back to disinfecting a rear side of a target.

9. The unmanned UAVD of claim 1, wherein the ozone generator comprises;
a casing configured to house a high velocity blower fan assembly with an adapter bracket for attaching the casing to the drone's body,
an elongated funnel shaped air delivery tube with its wider end attached to the exhaust side of the blower fan assembly,
a group of ceramic ozone generator plates installed inside the tube chamber;
a power supply configured to provide energy to power the ozone generator plates, an oxygen tank configured to supply extra oxygen to boost ozone production,
a color LED flashlight installed on a nozzle to guide the worker aiming the target to be blasting with ozone,
a handheld non-liquid sprayer configured to blast ozone at high velocity and long reaching disinfectant to disinfect viruses and to destroy farm pests with extra feed of attaching an oxygen tank to increase its potency and effectiveness,
a high pressurized air and ozone mix ejected at a nozzle of the tube shooting the ozone packs with high momentum impacting the virus hided in cracks,
a high pressurized air projecting ozone farther away from the operator reduces excessive ozone exposure.

10. The unmanned UAVD of claim 1, further comprising a free standing mobile robot configured for a non-aerial wide area disinfection further comprising;
a motorized screw drive shaft,
a motorized floor stand,
a system power battery,
an adapter bracket bridging the drone and the screw drive shaft of the free standing mobile robot, and
an upper end of a shaft urging toward a ceiling through a bumper and the lower end of the shaft attaching to a shaft motor supported by a motorized floor stand for a non-mobile operation.

11. The unmanned UAVD of claim 1, comprising a free-standing mobile robot configured for a non-aerial Upper-Room Germicidal UV-C disinfection system further comprising;
a motorized screw drive shaft,
a motorized floor stand,
a system power battery,
an adapter bracket bridging the drone and the screw drive shaft and a lower end of the shaft attaching to a shaft motor supported by a motorized floor stand of the free standing mobile robot,
a drone comprising a series of multi-purpose modules within the removable and configurable modular cartridge further comprising a fan assisted ozone generator module and an oscillation or rotation motion motorized hollow core tubular shaped cage module mounted with UV projectors,
a carriage lock via the screw drive shaft still allowing the drone to swing in a clockwise or a counter-clockwise direction parallel to the motorized screw drive shaft and allowing the drone to ascent or decent following a turning of threads of the screw drive shaft to better adjust different height of the room being disinfected, with a twist release of a lever to free the drone from the threads enabling the drone to slide along with the shaft at a desired elevation.

12. The unmanned UAVD of claim 1, further comprising an ozone decomposing face mask comprising;
a nose and a mouth covering made from non-woven face fabric,
an elastic ear-loop secure mouth covering fabric to wearer's ears,
an ozone decomposing box housing a check valve for exhale,
an activated carbon filter padding,
a removable and reusable stick of UV-C LED lighting powered by a rechargeable and reusable battery pack,
a reusable USB socket configured to attach to a battery pack to receive external power extending operation time of ozone decomposer,
an ozone rich air mixture enter through a short channel lined with activated carbon padding and a stick lined with a gang of UV-C LED,
a contaminated air slowed down to pass through the channel being irradiated with UV-C energy causing decomposing ozone to oxygen molecule, a decontaminated air pulled in at the end of channel via activated carbon padding and then exhaled through a check valve, and
an exhaled gas and fluid irradiated with UV-C LED energy in a check valve compartment prior to exit the face mask.

13. The unmanned UAVD of claim 12, comprising a solid state semiconductor projecting a unidirectional ultraviolet LED light configured to operate between a wavelength of 200 nm to 340 nm, each reusable UV LED die has a optically clear lumen enhancement such as a quartz lens potted on a stick and reusable components such as battery, the USB and the stick are removable and transferable to new decomposing box on face mask.

14. The unmanned UAVD of claim 1, comprising a free-standing mobile robot configured for a non-aerial isolation personal enclosed ozone disinfection in a booth system further comprising;
a motorized linear actuator drive shaft,
a motorized floor stand,
a system power battery,
an adapter bracket bridging the drone and the upper end of linear actuator drive shaft and the lower end of shaft attaching to a shaft motor supported by motorized floor stand of the free standing mobile robot,
an enclosed plastic panel booth with swing out door or lightweight pop-up enclosed canopy with zipper or magnetic strip door,
a rear-end ozone decomposition module having automatic power after a last person exited the system beyond a preset time for safeguarding visiting unmasked curious bystanders.

15. The Unmanned Aerial Vehicle Drone (UAVD) method, comprising:
Projecting zoom and focusable ultraviolet beam via optical lens and reflector onto disease infected target, tightly collimated UV beam concentrates in a low divergence spot striking harmful bacteria and virus causing destruction of their DNA;
controlling UV beam sweeps clockwise and counter-clockwise in-conjunction with ascent and decent motions of the drone irradiating the infected area on air and on surfaces;
positioning rotary shadow reflector bouncing back light beam on rear side of target;
controlling a communications of the UAVD via a controlling and a communications module comprising an electronic central processing unit (CPU), a wireless communication unit, an electronic camera and audio an A/V unit;
navigating the UAVD via a navigation module comprising a set of 360 degree obstacle avoidance sensors and positioning unit (GPS) configured to autonomously direct the drone to avoid obstacles while in flight; and
interconnecting the UV projector cage module, the control and communications module and the navigation module via a bus.

16. The method of claim 15, further comprising blasting the heavily disease infected area with ozone, using drone's propellers pulling in surrounding air blended with ozone configured to shower the target surfaces.

17. The method of claim 15, further comprising a non-aerial roaming on a ground level via a motorized floor stand with screw drive linear actuator carrying the drone sweeping the infected area with UV beams or showering with ozone for speedily disinfect the area,
in the event of a presence of occupants activate a front-end UV light and titanium oxide coated plates within the removable and configurable modular cartridge of the drone to disinfect the air via photocatalysis reaction, and
activate the UV beams in the hollow core tubular shaped cage module after Unmanned Aerial Vehicle Drone (UAVD) raises above the heads of room occupants before reaching the ceiling so its beams will clear the occupants via Upper-Room ultraviolet germicidal irradiation.

18. The method of claim 15, further comprising a non-aerial stationary disinfection in a booth blasting the mask protected occupant with high velocity turbulent air from the drone's propellers and blasting the occupant with high velocity ozone from any position to dislodge viruses for disinfection.

19. The method of claim 1, further comprising blasting agricultural pests such as caterpillars in farms with ozone causing damage to their respiratory organs for eradication.

20. The method of claim 1, further comprising removal of the ozone generator module from the drone, the removed ozone generator becomes a handheld ozone blaster operates by a worker for blasting ozone in high velocity to dislodge viruses from surfaces for disinfection and blasting farm pests with high boosted ozone for eradication.
